(19) United States
(12) Patent Application Publication
(10) Pub. No.: US 2010/0249882 A1
(43) Pub. Date: Sep. 30, 2010

(54) Acoustic Telemetry System for Communication with an Implantable Medical Device

(75) Inventor: Richard P.M. Houben, Lanaken (BE)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/415,542

(22) Filed: Mar. 31, 2009

(51) Int. Cl. A61N 1/08

(52) U.S. Cl. 607/60

(57) ABSTRACT

A telemetry module of an IMD operating in accordance with the techniques of this disclosure receives an unmodulated acoustic carrier signal from another device and modulates a reflected portion of the acoustic carrier signal with data for transmission to the other device. In one instance, the telemetry module may modulate the reflected portion of the carrier signal with data by selectively adjusting a reflectance of the transducer of the IMD. For example, the IMD may set the reflectance to be high or low depending on the information, e.g., digital 1 or 0, to be transmitted. The reflectance may be set high by leaving the electrical terminals of the transducer open such that there is no electrical dissipation and the reflectance may be set low by shorting the electrical terminals of the transducer such that energy is dissipated.

### TECHNICAL FIELD

The disclosure relates generally to implantable medical devices and, in particular, to an acoustic telemetry system for communication between an implantable medical device and another device.

### BACKGROUND

A wide variety of implantable medical devices (IMDs) that deliver a therapy to or monitor a physiologic or biological condition of a patient, or both, have been clinically implanted or proposed for clinical implantation in patients. The IMD may deliver therapy to or monitor a physiological or biological condition with respect to a variety of organs, nerves, muscles or tissues of the patients, such as the heart, brain, stomach, spinal cord, pelvic floor, or the like. The therapy provided by the IMD may include electrical stimulation therapy, drug delivery therapy or the like.

The IMD may exchange communications with another device. The IMD may exchange communications with an external device, such as a programming device or a monitoring device (e.g., either attached to the patient or otherwise located near the patient). Alternatively, or additionally, the IMD may communicate with another implantable device, e.g., another device that forms part of an intra-body communications network. The information exchanged may be physiological data acquired the IMD, information related to a therapy delivered by the IMD, or data indicating an operational status of the IMD. The IMD may also receive information from the programmer, such as configuration information that may be used to configure a therapy to be provided to the patient.

### SUMMARY

This disclosure relates to an acoustic telemetry system for communication between an IMD and another device, such as between an IMD and a non-implanted (or external) device or between two IMDs. The acoustic telemetry techniques of this disclosure are described in the context of communicating using ultrasound signals. However, the techniques may also be used in the context of other acoustic signals, such as sound signals or infrasound signals.

To communicate using ultrasound signals, each of the devices includes an ultrasound transducer that converts electrical signals into ultrasound signals and ultrasound signals into electrical signals. The ultrasound transducer of one of the devices, e.g., the external device for purposes of description, is driven by an electrical signal from a signal generator. The electrical signal is not modulated with any data. Instead, the ultrasound transducer of the external device converts the electrical signal into an unmodulated ultrasound carrier signal.

The ultrasound transducer of the IMD receives the unmodulated carrier signal from the external device and reflects at least a portion of the ultrasound carrier signal back to the external device. The IMD may modulate the reflected portion of the carrier signal with data for transmission to the external device. In one instance, the IMD may modulate the reflected portion of the carrier signal with data by selectively adjusting a reflectance of the ultrasound transducer of the IMD. For example, the IMD may set the reflectance to be high or low depending on the information, e.g., digital 1 or 0, to be transmitted. The reflectance may be set high by leaving the electrical terminals of the ultrasound transducer open such that there is no electrical dissipation and the reflectance may be set low by shorting the electrical terminals of the ultrasound transducer such that energy is dissipated.

In some instances, the IMD and the other device may communicate in at least two different ultrasound communication modes. The first ultrasound communication mode may be the ultrasound reflectance mode described above. The second ultrasound communication mode may be a direct pulsed ultrasound communication mode in which each of the devices transmits a modulated ultrasound signal. The direct pulsed communication mode may be particularly useful in instances in which bidirectional communication is desired, e.g., uplink and downlink communication with a programmer, or when it is not practical to drive one of the transducers with a continuous electrical signal, e.g., for intra-body communication between two IMDs.

In one example, this disclosure is directed to an implantable medical device comprising an ultrasound transducer that receives an ultrasound carrier signal incident on the ultrasound transducer and a control unit that selectively adjusts a reflectance of the ultrasound transducer to modulate a reflected portion of the ultrasound carrier signal with data to be transmitted from the implantable medical device.

In another example, this disclosure is directed to a method comprising receiving an ultrasound carrier signal incident on an ultrasound transducer of an implantable medical device and modulating a reflected portion of the ultrasound carrier signal with data to be transmitted from the implantable medical device.

In another example, this disclosure is directed to an implantable medical device comprising means for receiving an ultrasound carrier signal and means for modulating a reflected portion of the ultrasound carrier signal with data to be transmitted from the implantable medical device.

This summary is intended to provide an overview of the subject matter described in this disclosure. It is not intended to provide an exclusive or exhaustive explanation of the invention as described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF THE DRAWINGS

FIG. 1 is a conceptual diagram illustrating an example medical system in which the devices communicate using at least the ultrasound telemetry techniques described in this disclosure.

FIG. 2 is a block diagram illustrating an example IMD and external device in further detail.

FIG. 3 is a schematic diagram illustrating an example telemetry module that modulates a reflected portion of an incident carrier signal to send data.

FIG. 4 is a schematic diagram illustrating an example telemetry module in further detail.

FIG. 5 is a schematic diagram illustrating an example telemetry module.

FIG. 6 is a block diagram illustrating an example telemetry module that operates in accordance with the ultrasound reflectance communication techniques of this disclosure.

FIG. 7 is a block diagram illustrating another example telemetry module.

FIG. 8 is a flow diagram illustrating example operation of a telemetry module of an IMD operating in accordance with the ultrasound reflectance communication mode.

FIG. 9 is a flow diagram illustrating example operation of an IMD communicating using ultrasound communication in accordance with one aspect of this disclosure.

### DETAILED DESCRIPTION

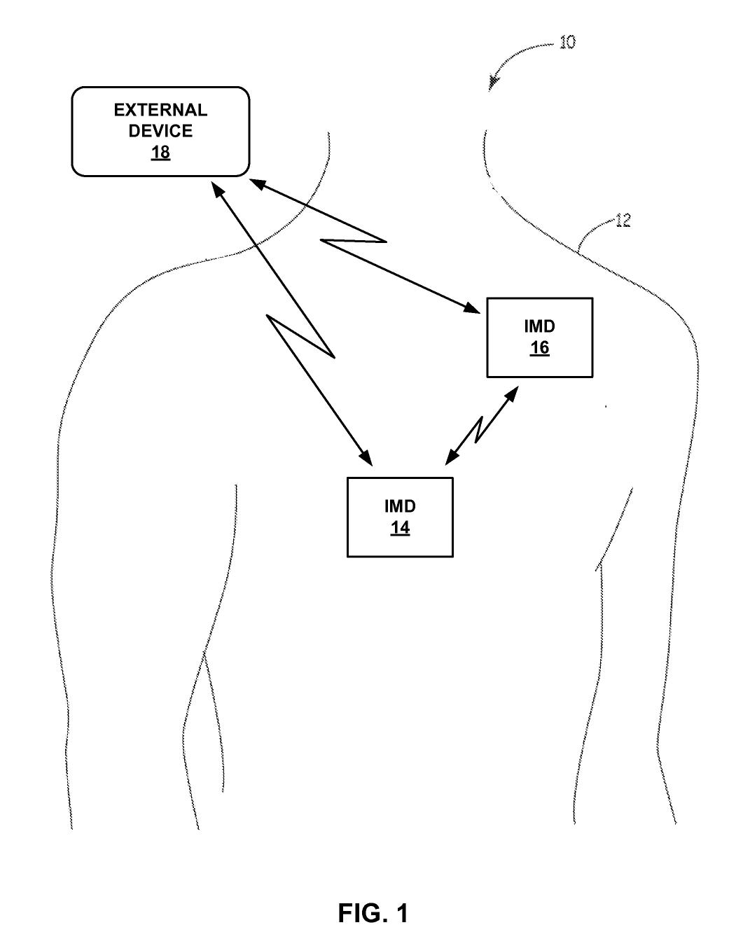

FIG. 1 is a conceptual diagram illustrating an example medical system 10 in which the devices communicate using at least the acoustic telemetry techniques described in this disclosure. The acoustic telemetry techniques of this disclosure are described in the context of communicating using ultrasound signals. However, the techniques may also be used in the context of other acoustic signals.

The medical devices of medical system 10 may include one or more medical devices that may be used to provide therapy to and/or sense one or more physiological signals of a patient 12. The medical devices of medical system 10 may also include devices that interact with IMDs to program the IMDs and/or retrieve date from the IMDs, such as programming devices and/or monitoring devices. In the example illustrated in FIG. 1, medical system 10 includes an IMD 14, IMD 16, and external (or non-implanted) device 18. Medical system 10 may, however, include more or fewer medical devices that may or may not be implanted within patient 12.

IMD 14 may be any of a variety of medical devices that provide therapy to patient 12, sense physiological or biological conditions of patient 12 or a combination thereof. In some instances, IMD 14 may be a device that provides electrical stimulation therapy in the form of cardiac rhythm management therapy to a heart of patient 12. In such a case, IMD 14 may include one or more implantable leads (not shown) with one or more electrodes that extend from IMD 14 for delivering therapy to and/or sensing physiological signals of a heart of patient 12. The leads may be implanted within one or more atria or ventricles of the heart of patient 12 or a combination thereof. In other words, IMD 14 may be used for single chamber or multi-chamber cardiac rhythm management therapy. The cardiac rhythm management therapy delivered by IMD 14 may include, for example, pacing, cardioversion, defibrillation and/or cardiac resynchronization therapy (CRT). In other instances, IMD 14 may be a device that provides electrical stimulation to a tissue site of patient 12 proximate a muscle, organ or nerve, such as a tissue proximate a vagus nerve, spinal cord, brain, stomach, pelvic floor or the like to treat various conditions, including movement and affective disorders such as chronic pain, Parkinson's disease, tremor and dystonia, urinary storage and voiding dysfunction, digestion dysfunction, sexual dysfunction or the like.

Alternatively, IMD 14 may be a device that delivers a drug or therapeutic agent to patient 12 via an implantable catheter (not shown). IMD 14 may, for example, be implanted within a subcutaneous pocket in an abdomen of patient 12 and the catheter may extend from IMD 14 into the stomach, pelvic floor, brain, intrathecal space of the spine of patient 12 or other location depending on the application. IMD 14 may deliver the drug or therapeutic agent via the catheter to reduce or eliminate the condition of the patient and/or one or more symptoms of the condition of the patient. For example, IMD 14 may deliver morphine or ziconotide to reduce or eliminate pain, baclofen to reduce or eliminate spasticity, chemotherapy to treat cancer, or any other drug or therapeutic agent to treat any other condition and/or symptom of a condition.

Like IMD 14, IMD 16 may also be any of a variety of implantable medical devices that sense a physiological or biological condition of and/or deliver therapy to patient 12. As one example, IMD 16 may be a wireless (or leadless) sensor implanted within patient 12 to sense one or more physiological signals of patient 12. IMD 16 may be implanted at targeted monitoring sites and transmit the sensed signals, thus avoiding limitations associated with lead-based sensors. In some instances, IMD 16 uses the sensed physiological signals to monitor a condition of patient 12 or provide therapy to patient 12 as a function of the sensed physiological signals. Alternatively, or additionally, IMD 16 transmits the sensed physiological signals to another device, such as IMD 14 or external device 18, which may in turn monitor the condition of patient 12 or provide therapy to patient 12 as a function of the sensed physiological signals. IMD 16 may sense, sample, and process one or more physiological signals such as heart activity, muscle activity, brain electrical activity, intravascular pressure, blood pressure, blood flow, acceleration, displacement, motion, respiration, or blood/tissue chemistry, such as oxygen saturation, carbon dioxide, pH, protein levels, enzyme levels or other parameter.

Although IMD 16 is described with reference to FIG. 1 as being a wireless sensor, IMD 16 may be any of a variety of other medical devices that deliver therapy, sense physiological signals or both. For example, IMD 16 may be a leadless pacer (sometimes referred to as a wireless pacer). Other examples of medical devices that IMD 16 could be include therapy delivery devices, such as electrical stimulation devices that deliver electrical stimulation to a heart, brain, spinal cord, stomach, pelvic floor or other location within or on patient 12, or drug pumps or infusion pumps that delivers a drug, therapeutic agent, saline solution, or other liquid to locations within patient 12.

External device 18 may be a programming device or monitoring device that allows a user, e.g., physician, clinician or technician, to configure a therapy delivered by IMDs 14 and/or 16 or to retrieve data sensed by IMDs 14 and/or 16. External device 18 may include a user interface that receives input from the user and/or displays data to the user, thus allowing the user to program the therapy delivered by IMDs 14 and/or 16 or display data retrieved from IMDs 14 and/or 16. External device 18 may be a dedicated hardware device with dedicated software for programming or otherwise communicating with IMDs 14 and/or 16. Alternatively, external device 18 may be an off-the-shelf computing device running an application that enables external device 18 to program or otherwise communicate with IMDs 14 and/or 16. In some examples, external device 18 may be a handheld computing device that may be attached to or otherwise carried by patient 12. Alternatively, external device 18 may be a computer workstation, such as a CareLink® monitor, available from Medtronic, Inc. of Minneapolis, Minn.

IMD 14, IMD 16 and external device 18 wirelessly communicate with one another. In some instances, IMD 14, IMD 16 and external device 18 may be communicatively coupled with each other as well as other medical devices (not shown) to form a local area network, sometimes referred to as a body area network (BAN) or personal area network (PAN). Each device may therefore be enabled to communicate wirelessly along multiple pathways with each of the other networked devices. As such, IMD 14, IMD 16 and external device 18 may represent a distributed system of implantable medical devices that cooperate to monitor a condition of and/or provide therapy to patient 12.

IMD 14, IMD 16 and external device 18 may wirelessly communicate with one another using the acoustic telemetry techniques of this disclosure. Although described below in the context of communicating using ultrasound signals, the techniques may also be used in the context of other acoustic signals. Additionally, for purposes of illustration, the ultrasound telemetry techniques will be described in the context of external device 18 communicating with IMD 16. However, the ultrasound telemetry techniques of this disclosure may be used for communication between external device 18 and IMD 14 or between IMD 14 and IMD 16.

External device 18 includes an ultrasound transducer that transmits an unmodulated ultrasound carrier signal to IMD 16. In other words, the carrier signal transmitted to IMD 16 is not modulated with outbound data from external device 18. IMD 16 includes an ultrasound transducer that receives the unmodulated ultrasound carrier signal incident on the transducer, and reflects at least a portion of the ultrasound carrier signal back to external device 18. IMD 16 modulates the reflected portion of the ultrasound carrier signal with outbound data for transmission to external device 18. In one example, IMD 16 selectively adjusts the reflectance of the ultrasound transducer to modulate the reflected portion of the carrier signal with the outbound data. In other words, IMD 16 may selectively adjust the reflectance of the ultrasound transducer to be high or low depending on the information, e.g., digital 1 or 0, to be transmitted. In this manner, IMD 16 amplitude modulates the reflected portion of the ultrasound carrier signal. External device 18 receives the reflected portion of the carrier signal that has been amplitude modulated with the data from IMD 16. The reflected portion of the carrier signal may, in other instances, be modulated using other types of modulation, such as frequency or phase modulation, as described in more detail below.

In some instances, IMD 16 needs to receive data, e.g., as in the case of programming IMD 16 or it is not practical to drive a transducer of one of the IMDs with a continuous electrical signal, e.g., as in the case of intra-body communication between IMD 14 and IMD 16. In these instances, IMD 14, IMD 16 and/or external device 18 may communicate using at least two different ultrasound communication modes. The first ultrasound communication mode may be the ultrasound reflectance mode described above in which the reflected portion of the carrier signal is modulated, e.g., by selectively adjusting a reflectance of the ultrasound transducer of IMD 14 and/or IMD 16. The second ultrasound communication mode may be a direct pulsed ultrasound communication mode. In the direct pulsed communication mode, external device 18 modulates a carrier signal with the outgoing data and transmits the modulated carrier signal via the ultrasound transducer. IMD 16 receives the modulated carrier signal with its ultrasound transducer and demodulates the carrier signal to obtain the data from external device 18. In the direct pulsed communication mode, IMD 16 may also generate a carrier signal, modulate the carrier signal with the outgoing data and transmit the data via the ultrasound transducer to external device 18, which demodulates the carrier signal to obtain the data.

In another example, IMD 16 and external device 18 may communicate using a full duplex communication channel in which communications from external device 18 to IMD 16 are modulated using a first modulation technique and the communications from IMD 16 to external device 18 are modulated using the reflectance mode with a second modulation technique that is different than the first modulation technique.

In addition to ultrasound telemetry, IMD 14, IMD 16 and external device 18 may communicate using radio frequency (RF) telemetry. In one instance, IMD 14, IMD 16 and/or external device 18 may communicate in accordance with the Medical Implant Communications Service (MICS) band regulation or the Medical External Data Service (MEDS) band regulation. As such, IMD 14, IMD 16 and external device may include appropriate modulation, demodulation, frequency conversion, filtering, amplifier, and antenna components for transmission and reception of data via RF.

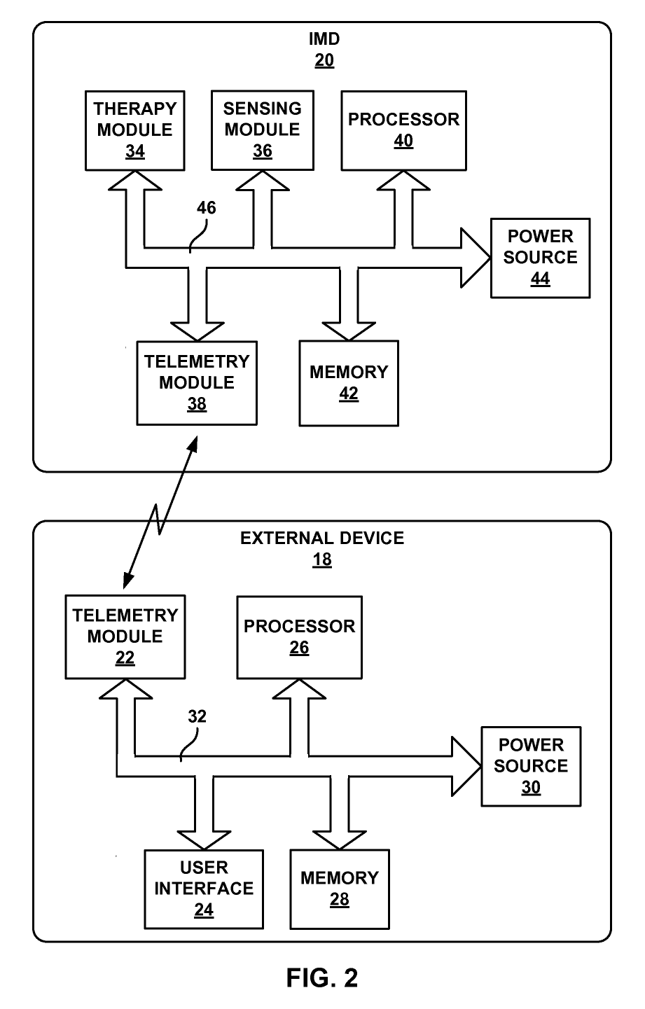

FIG. 2 is a block diagram illustrating an example IMD 20 and external device 18 in further detail. IMD 20 may correspond to IMD 14 or IMD 16 of FIG. 1, or another IMD. External device 18 may correspond to a programming device, a monitoring device or other external device located on or in the vicinity of patient 12. As illustrated in the example of FIG. 2, external device 18 includes a telemetry module 22, user interface 24, processor 26, memory 28 and power source 30, all of which are interconnected by a data bus 32. IMD 20 includes a therapy module 34, sensing module 36, telemetry module 38, processor 40, memory 42 and power source 44, all of which are interconnected by a data bus 46.

The various components of IMD 20 are coupled to power source 44, which may include a rechargeable or non-rechargeable battery. A non-rechargeable battery may be selected to last for several years, while a rechargeable battery may be charged from an external charging device on a daily or weekly basis. In either case, and especially in the case of the non-rechargeable battery, the amount of power of the battery is limited. Alternatively, power source 44 may be another energy storage device, and energy harvesting device, or a combination of a rechargeable or non-rechargeable battery, an energy harvesting device and energy storage device, or other type of power source.

IMD 20 may sense one or more physiological signals or conditions of patient 12. In some instances, IMD 20 may not provide therapy to patient 12, but only provide monitoring of patient 12 as in the case of an implantable loop recorder. In such cases, IMD 20 may not include therapy module 34. Sensing module 36 is configured to monitor one or more physiological signals using one or more sensors connected to sensing module 36. In one example, sensing module 36 is configured to monitor signals sensed by one or more of electrodes on leads extending from IMD 20. In another example, sensing module 36 may be configured to monitor signals sensed by a sensor within or on IMD 20. In a further example, sensing module 36 may be configured to receive signals sensed by one or more wireless or lead-less sensors and transmitted wirelessly to IMD 20. The one or more sensors may sense physiological signals such as heart activity (e.g., electrocardiogram (ECG) signals), muscle activity (e.g., electromyography (EMG) signals), brain electrical activity (e.g., electroencephalography (EEG) signals), heart rate, intravascular pressure, blood pressure, blood flow, acceleration, displacement, motion, respiration, or blood/tissue chemistry such as oxygen saturation, carbon dioxide, pH, protein levels, enzyme levels or other parameter.

Sensing module 36 may store the sensed signals in memory 42. In some instances, sensing module 36 may store the sensed signals in raw form. In other instances, sensing module 36 may process the sensed signals and store the processed signals in memory 42. For example, sensing module 36 may amplify and filter the sensed signal and store the filtered signal in memory 42. The signals stored by sensing module 36 may, in some cases, be retrieved and further processed by processor 40.

IMD 20 may also provide therapy, such as electrical stimulation therapy or drug delivery therapy, to patient 12 in accordance with parameters of one or more selected therapy programs. In particular, processor 40 controls therapy module 34 to deliver therapy to patient 12 according to one or more therapy programs, which may be received from external device 18 and stored in memory 42. In the case of electrical stimulation therapy, therapy module 34 may include a stimulation generator that generates and delivers electrical stimulation therapy, e.g., in the form of pulses or shocks. Processor 40 may control the stimulation generator to deliver electrical stimulation pulses with amplitudes, pulse widths, frequency, and/or electrode polarities specified by the one or more therapy programs. In the case of drug delivery therapy, therapy module 34 may include a pump that delivers a drug or therapeutic agent to patient 12. Processor 40 may control the pump to deliver the drug or therapeutic agent with the dosage and frequency (or rate) specified by the one or more therapy programs.

Processor 40 may include any one or more of a microprocessor, a controller, a digital signal processor (DSP), an application specific integrated circuit (ASIC), a field-programmable gate array (FPGA), or equivalent discrete or integrated logic circuitry. In some examples, processor 40 may include multiple components, such as any combination of one or more microprocessors, one or more controllers, one or more DSPs, one or more ASICs, or one or more FPGAs, as well as other discrete or integrated logic circuitry. The functions attributed to processor 40 herein may be embodied as software, firmware, hardware or any combination thereof.

Memory 42 includes computer-readable instructions that, when executed by processor 40, cause IMD 20 and processor 40 to perform various functions attributed to IMD 20 and processor 40 herein. Memory 42 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, magnetoresistive random access memory (MRAM), or any other digital media.

A user may interact with external device 18 to retrieve data from IMD 20. The data retrieved from IMD 20 may include real-time or stored physiological data acquired by IMD 20, diagnosis data generated based on the acquired physiological data, therapy history data stored by IMD 20, data indicating an operational status of IMD 20 (e.g., remaining battery power or lead integrity), or other type of data stored by IMD 20. User interface 24 may include, for example, a keypad and a display, which may be, for example, a cathode ray tube (CRT) display, a liquid crystal display (LCD) or light emitting diode (LED) display. The keypad may take the form of an alphanumeric keypad or a reduced set of keys associated with particular functions. External device 18 can additionally or alternatively include a peripheral pointing device, such as a mouse, via which a user may interact with the user interface. In some embodiments, the display of external device 18 may include a touch screen display, and a user may interact with external device 18 via the display.

In response to input from the user, processor 26 controls telemetry module 22 to retrieve the data from IMD 20. For example, processor 26 may enable transmit and receive circuitry of telemetry module 22 in response to receiving a command from a user to interrogate IMD 20. Telemetry module 22 may transmit and receive communications with telemetry module 38 using ultrasound signals or other acoustic signals. Telemetry module 22 includes any suitable hardware, firmware, software or any combination thereof for communicating with another device, such as external device 18, using acoustic signals. For example, telemetry module 38 may include one or more acoustic (or ultrasound) transducers, signal generators, modulators, demodulators, frequency converters, filters or amplifiers for transmission and reception of data via ultrasound signals.

For example, an ultrasound transducer of telemetry module 22 converts electrical signals into ultrasound signals for transmission to telemetry module 38 of IMD 20 and converts ultrasound signals received from telemetry module 38 of IMD 20 into electrical signals. A signal generator of telemetry module 22 may drive the ultrasound transducer with an unmodulated electrical signal. In one example, the signal generator drives the ultrasound transducer with a continuous electrical signal, such as a continuous sine wave. The continuous electrical signal is not modulated with any data for transmission to IMD 20. The ultrasound transducer of telemetry module 22 converts the continuous electrical signal into an unmodulated ultrasound carrier signal and transmits the unmodulated ultrasound carrier signal to telemetry module 38 of IMD 20. Alternatively, the ultrasound transducer may be driven with a modulated signal that is modulated using a different modulation technique than the reflected portion of the carrier signal.

Telemetry module 38 of IMD 20 includes an ultrasound transducer that receives the unmodulated ultrasound carrier signal from telemetry module 22 of external device 18. At least a portion of the ultrasound carrier signal is reflected back to external device 18. The amount of the ultrasound carrier signal reflected depends on the reflectance of the ultrasound transducer of IMD 20. A larger reflectance results in a larger portion of the carrier signal being reflected back to external device 18. In accordance with the techniques of this disclosure, telemetry module 38 may modulate the reflected portion of the carrier signal with data for transmission to the external device 18. In one instance, telemetry module 38 may selectively adjust the reflectance of the ultrasound transducer of telemetry module 38 to modulate the reflected portion of the carrier signal with data for transmission. For example, telemetry module 38 may set the reflectance of the ultrasound transducer to be high or low depending on the information, e.g., digital 1 or 0, to be transmitted. A high reflectance may represent a digital 1 and a low reflectance may represent a digital 0, or the other way around, i.e., a high reflectance may represent a digital 0 and a low reflectance may represent a digital 1.

Telemetry module 38 may produce a high reflectance by leaving the electrical terminals of the ultrasound transducer “open.” When the electrical terminals of the ultrasound transducer are open, there is no electrical dissipation thus resulting in a high reflectance. Alternatively, telemetry module 38 may produce a high reflectance by applying a large resistive load across the electrical terminals of the ultrasound transducer. Telemetry module 38 may produce a low reflectance by shorting the electrical terminals of the ultrasound transducer or applying a resistive load (smaller than the resistive load for generating the high reflectance) across the electrical terminals of the ultrasound transducer. In either case, energy is dissipated to produce a lower reflectance than when the terminals are open or coupled to the larger resistive load. IMD 20 may selectively adjust the reflectance of the ultrasound transducer based on the data to be transmitted.

External device 18 receives the reflected portion of the carrier signal that has been modulated with the data from IMD 20. A signal detector of external device 18 may detect the reflected portion of the carrier signal by analyzing the output of the transducer of external device 18. In other instances, external device may include a separate receive transducer and monitor the electrical output of the receive transducer. External device 18 may also include a demodulator that demodulates the modulated signal to obtain the data from IMD 20. Processor 26 may store the retrieved data in memory 28 for later processing or later transmission to another device, such as a remote server.

As such, the ultrasound reflectance communication techniques provide a unidirectional communication method in which data is transferred from the IMD 20 to the external device 18 with relatively little power consumption within IMD 20. The relatively little power consumption within IMD 20 extends the service life of power source. In some instances, it may be desirable to have bidirectional communication between external device 18 and IMD 20. For example, bidirectional communication may be desired for configuring operation of IMD 20, such as programming IMD 20 to provide therapy in accordance with a selected therapy program, programming IMD 20 to acquire physiological data, and/or programming IMD 20 to perform one or more diagnostic tests (e.g., battery life test or lead integrity test). In other instances, it is not practical to drive one of the transducers with a continuous electrical signal. In the case of intra-body communication between IMD 20 and another IMD, it is not practical to drive a transducer of one of the IMDs with a continuous electrical signal due to the limited supply of power.

As such, IMD 20 and external device 18 may be capable of communicating in accordance with at least two different ultrasound communication modes. The first ultrasound communication mode may be the ultrasound reflectance mode described in detail above. The second ultrasound communication mode may be a direct pulsed ultrasound communication mode in which each of telemetry modules 22 and 38 transmits a modulated ultrasound signal. For example, telemetry module 22 of external device 18 may generate a carrier signal, modulate the carrier signal with outgoing data and transmit the modulated carrier signal via the ultrasound transducer. Telemetry module 38 of IMD 20 receives the modulated carrier signal with its ultrasound transducer, demodulates the carrier signal to obtain the data from external device 18 and provides the data to processor 40, which may process the data or store the data in memory 42 for later processing.

In the direct pulsed ultrasound mode, telemetry module 38 of IMD 20 may also generate a carrier signal, modulate the carrier signal with outbound data, and drive the ultrasound transducer of telemetry module 38 with the modulated electrical carrier signal to produce a modulated ultrasound carrier signal for transmission to external device 18. In this manner, the direct pulsed communication mode enables bidirectional communication with the other device. Thus, in some instances, telemetry modules 22 and 38 may transmit and receive data in the direct pulsed mode. This may be the case when performing intra-body communication between IMD 20 and another IMD.

In other instances, telemetry modules 22 and 38 may operate in the direct pulsed mode only for communicating data to IMD 20. For example, telemetry modules 22 and 38 may operate in the direct pulsed communication mode when transmitting data from external device 18 to IMD 20 and operate in the reflectance communication mode when transmitting data from IMD 20 to external device 18. This may be the case when programming IMD 20 with external device 18.

In any case, IMD 20 and external medical device 18 may switch ultrasound communication modes in response to a command from the user received via user interface 24. In some instances, the user selects a particular communication mode, e.g., unidirectional or bidirectional communication mode. In other instances, the user may enter a command to perform some action, and processor 26 of external device 18 may determine the communication mode based on the action to be performed. For example, processor 26 may configure telemetry module 22 to use the direct pulsed mode in response to the user entering a command to program IMD 20. As another example, processor 26 may configure telemetry module 22 to use the ultrasound reflectance mode in response to the user entering a command to download data from IMD 20. External device 18 may send a communication to IMD 20 that specifies the particular ultrasound communication mode.

In other instances, IMD 20 and external device 18 may communicate using a full duplex communication channel in which communications from external device 18 to IMD 20 (download communications) are modulated using a first modulation technique and the communications from IMD 20 to external device 18 (upload communications) are modulated using the reflectance mode with a second modulation technique that is different than the first modulation technique. For example, external device 18 may generate a carrier signal and modulate the carrier signal using frequency modulation. IMD 20 may receive the frequency modulated signal and demodulate the signal to obtain the data transmitted by external device 18. The transducer of IMD 20 reflects a portion of the carrier signal incident on the transducer. IMD 20 may adjust the reflectance of the transducer to amplitude modulate the reflected portion of the carrier signal. Thus, the reflected portion of the carrier signal may be modulated concurrently with the demodulation of the received portion of the carrier signal. In other examples, upload communications may be modulated using phase modulation and download communication may be modulated using amplitude modulation, upload communications may be modulated using amplitude modulation and download communication may be modulated using frequency modulation, or other combinations of modulation techniques.

As described above, the ultrasound reflectance communication techniques provide a communication technique in which data is transferred from the IMD 20 to the external device 18 with relatively little power consumption within IMD 20. Additionally, both the ultrasound communication techniques are not sensitive to electromagnetic interference, e.g., as caused by MRI or other interference.

Processor 26 may include one or more of a microprocessor, a controller, a DSP, an ASIC, a FPGA, or equivalent discrete or integrated logic circuitry. In some examples, processor 26 may include multiple components, such as any combination of one or more microprocessors, one or more controllers, one or more DSPs, one or more ASICs, or one or more FPGAs, as well as other discrete or integrated logic circuitry. The functions attributed to processor 26 herein may be embodied as software, firmware, hardware or any combination thereof.

Memory 28 includes computer-readable instructions that, when executed by processor 26, cause external device 18 and processor 26 to perform various functions attributed to external device 18 and processor 26 herein. Memory 28 may include any volatile, non-volatile, magnetic, optical, or electrical media, such as a RAM, ROM, NVRAM, EEPROM, flash memory, MRAM, or any other digital media.

Power source 30 of external device 18 delivers operating power to the components of external device 18. Power source 30 may include a battery and a power generation circuit to produce the operating power for the components of external device 18. In some examples, the battery may be rechargeable (e.g., nickel cadmium or lithium ion batteries) to allow extended operation. Recharging may be accomplished by electrically coupling power source 30 to a cradle or plug that is connected to an alternating current (AC) outlet. In addition or alternatively, recharging may be accomplished through proximal inductive interaction between an external charger and an inductive charging coil within external device 18. In other embodiments, non-rechargeable batteries (e.g., non-rechargeable lithium based batteries such as lithium iodide) may be used. In addition, external device 18 may be directly coupled to an AC outlet to power external device 18.

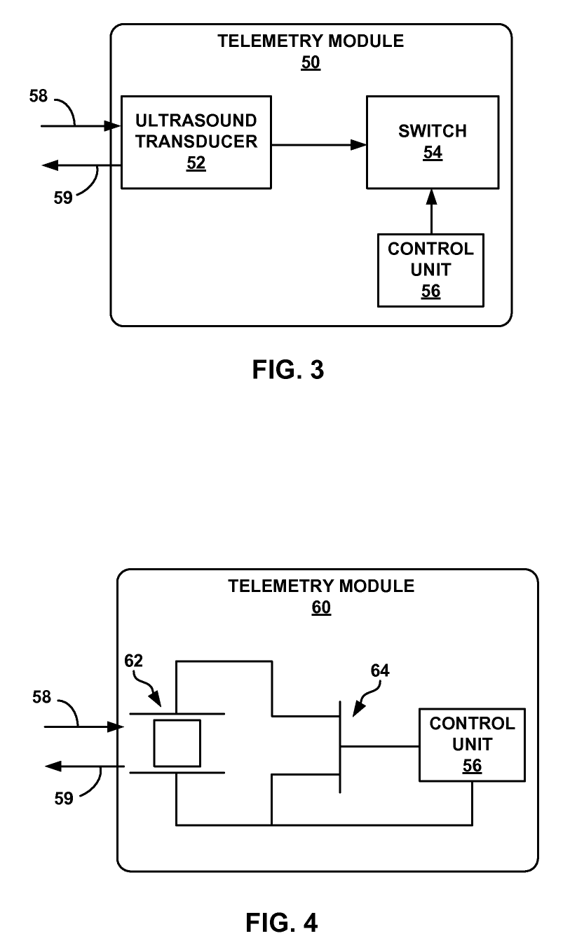

FIG. 3 is a schematic diagram illustrating an example telemetry module 50 that modulates a reflected portion of an incident carrier signal to send data. Telemetry module 50 may correspond to telemetry module 38 of IMD 20 illustrated in FIG. 2 or a telemetry module of a different device. Telemetry module 50 includes an ultrasound transducer 52, a switch 54 and a control unit 56.

An unmodulated carrier signal transmitted from another device, such as external device 18, is incident on ultrasound transducer 52. The unmodulated carrier signal incident on ultrasound transducer 52 is represented by arrow 58. A portion of the carrier signal incident on ultrasound transducer 52 is reflected back to external medical device 18. The reflected portion of the carrier signal is represented by arrow 59. As described above, the amount of the carrier signal reflected by ultrasound transducer 52 depends on the reflectance of ultrasound transducer 52. In accordance with one aspect of this disclosure, telemetry module 50 selectively adjusts the reflectance of ultrasound transducer 52 to modulate the reflected portion of the carrier signal with outbound data for transmission to external device 18.

Control unit 56 may selectively open and close switch 54 to adjust the reflectance of ultrasound transducer 52. Switch 54 may be any of a number of components or devices that have one state in which current is conducted through switch 54 and a second state in which current is not conducted through switch 54 thereby breaking the electrical circuit. In one example, switch 54 is a transistor, such as a field-effect transistor (FET) or bipolar junction transistor (BJT). In other examples, switch 54 may be an electromechanical switch, such as a microelectromechanical system (MEMS) switch.

Switch 54 may be connected between electrical terminals of ultrasound transducer 52. Current flows through switch 54 when switch 54 is closed, thereby short circuiting the electrical terminals of ultrasound transducer 52. The short circuiting of the electrical terminals of ultrasound transducer 52 produces a large current dissipation that results in ultrasound transducer 52 having a low reflectance. When switch 54 is open, no current or a negligible amount of current flows through switch 54. As such, the electrical terminals of ultrasound transducer 54 appear as on open circuit with no current dissipation, resulting in ultrasound transducer 52 having a high reflectance.

As described above, the amount of the incident ultrasound carrier signal that is reflected by ultrasound transducer 52 depends on the reflectance of ultrasound transducer 52. In other words, the amplitude of the reflected signal is higher when ultrasound transducer 52 has a large reflectance and smaller when ultrasound transducer 52 has a low reflectance. Control unit 56 may therefore open and close switch 54 to selectively adjust the reflectance of ultrasound transducer 52 to amplitude modulate the reflected portion of the incident carrier signal. In one example, control unit 56 closes switch 54 when the data to be transmitted is a “1” and opens switch 54 when the data to be transmitted is a “0.” In this case, the reflected portion of the carrier signal is amplitude modulated such that the reflected signal has a smaller amplitude to represent a “1,” e.g., due to the low reflectance of transducer 52 when switch 54 is closed, and a larger amplitude to represent a “0,” e.g., due to the high reflectance of transducer 52 when the switch is open. In another example, control unit 56 opens switch 54 when the data to be transmitted is a “0” and closes switch 54 when the data to be transmitted is a “1.” As such, the reflected signal may be viewed as being amplitude modulated with the data to be transmitted.

In other instances, switch 54 may couple one or more resistive loads (not shown) or other loads to the electrical terminals of ultrasound transducer 52. For example, switch 54 may be electrically coupled to a load across the electrical terminals of ultrasound transducer 52 such that when the switch is open no load is applied across the electrical terminals of transducer 52 resulting in a high reflectance and when the switch is closed a resistive or other load is placed across the electrical terminals of transducer 52 resulting in a lower reflectance than when the switch is open. As another example, switch 54 may electrically couple different resistive loads across the electrical terminals of transducer 52 where one of the resistive loads results in transducer 52 having a higher reflectance than the other resistive load.

Alternatively, telemetry module 50 may modulate the reflected portion of the carrier signal using other types of modulation, e.g., frequency or phase modulation. For example, switch 54 may couple one or more non-resistive loads (e.g., capacitive, inductive or a combination thereof) to the electrical terminals of ultrasound transducer 52. When switch 54 is open, no load is applied across the electrical terminals of transducer 52 and the transducer is tuned to a first frequency. When switch 54 is closed, the non-resistive load is placed across the electrical terminals of transducer 52 resulting in transducer 52 being tuned to a second frequency. The second frequency is different than the first frequency and may be either higher or lower than the first frequency. In one example, control unit 56 closes switch 54 when the data to be transmitted is a “0” and opens switch 54 when the data to be transmitted is a “1.” In this case, the reflected portion of the carrier signal has the first frequency when the data to be transmitted is a “1” and the reflected portion of the carrier signal has the second frequency when the data to be transmitted is a “b 0.”

Control unit 56 may include any one or more of a microprocessor, a controller, a digital signal processor (DSP), an application specific integrated circuit (ASIC), a field-programmable gate array (FPGA), or equivalent discrete or integrated logic circuitry. In some examples, control unit 56 may include multiple components, such as any combination of one or more microprocessors, one or more controllers, one or more DSPs, one or more ASICs, or one or more FPGAs, as well as other discrete or integrated logic circuitry. The functions attributed to control unit 56 herein may be embodied as software, firmware, hardware or any combination thereof.

FIG. 4 is a schematic diagram illustrating an example telemetry module 60 in further detail. Telemetry module 60 may correspond to telemetry module 50 of FIG. 3 with ultrasound transducer 52 being a piezoelectric transducer 62 and switch 54 being a transistor 64. In accordance with one aspect of this disclosure, control unit 56 selectively adjusts the reflectance of piezoelectric transducer 62 by turning transistor 64 ON and OFF to modulate the reflected portion of the carrier signal with data for transmission to external device 18.

When a voltage supplied by control unit 56 between a gate and drain of transistor 64 is greater than or equal to a threshold voltage, transistor 64 is turned ON. In the ON state (sometimes referred to as a saturation state), transistor 64 operates in the same manner as if a switch has been closed and current flows from the source to the drain. Thus, transistor 64 may be viewed as approximating a short circuit between the electrical terminals of piezoelectric transducer 62 in the ON state. This results in piezoelectric transducer 62 having a low reflectance.

When the voltage supplied by control unit 56 between the gate and drain of transistor 64 is less than the threshold voltage, transistor 64 is turned OFF. In the OFF state, transistor 64 operates in the same manner as if a switch has been opened and no current (or a small amount of current) flows from the source to the drain. Thus, transistor 64 may be viewed as approximating an open circuit between the electrical terminals of piezoelectric transducer 62 in the OFF state. This results in piezoelectric transducer 62 having a high reflectance.

Control unit 56 may generate the control voltage supplied to transistor 64 based on the data to be transmitted, which may be received from processor 40 (FIG. 2). In this manner, control unit 56 selectively turns transistor 64 ON and OFF based on the data to be transmitted. In one example, control unit 56 turns transistor 64 ON when the data to be transmitted is a “1” and turns transistor 64 OFF when the data to be transmitted is a “0.” In this case, the reflected portion of the carrier signal is amplitude modulated such that the reflected signal has a smaller amplitude to represent a “1,” e.g., due to the low reflectance of transducer 62 when transistor 64 is ON, and a larger amplitude to represent a “0,” e.g., due to the high reflectance of transducer 62 when transistor 64 is OFF. In another example, control unit 56 turns transistor 64 ON when the data to be transmitted is a “0” and turns transistor 64 OFF when the data to be transmitted is a “1.” In this case, the reflected portion of the carrier signal is amplitude modulated such that the reflected signal has a larger amplitude to represent a “1,” e.g., due to the high reflectance of transducer 62 when transistor 64 is OFF, and a smaller amplitude to represent a “0,” e.g., due to the low reflectance of transducer 62 when transistor 64 is ON. In other instances, the data to be transmitted may be applied directly to the gate of transistor 64 to turn transistor 64 ON and OFF. In this case, processor 40 may be viewed as the control unit and the data may be viewed as the control voltage supplied to transistor 64.

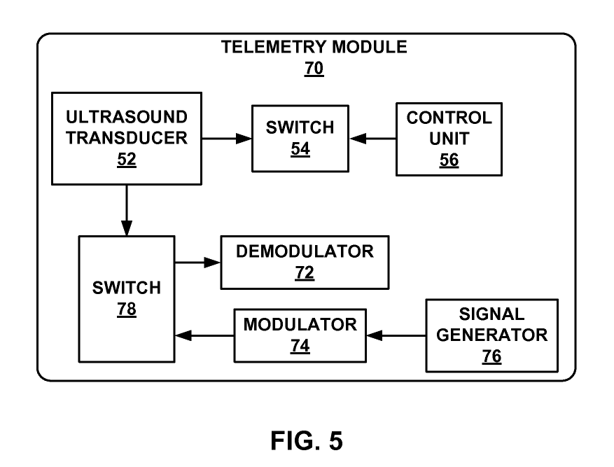

FIG. 5 is a schematic diagram illustrating an example telemetry module 70. Telemetry module 70 may correspond to telemetry module 38 of IMD 20 illustrated in FIG. 2 or a telemetry module of a different device. Telemetry module 70 is capable of operating in at least two different ultrasound communication modes depending on the application to provide bidirectional ultrasound communication.

Telemetry module 70 of FIG. 5 includes an ultrasound transducer 52, a switch 54, a control unit 56, a demodulator 72, a modulator 74, a signal generator 76 and a switch 78. Switch 78 is opened and closed to configure the ultrasound communication mode of telemetry module 70. Telemetry module 70 operates in a reflectance ultrasound communication mode when switch 78 is open and operates in a direct pulsed ultrasound communication mode when switch 78 is closed. Control unit 56 opens switch 54 while switch 78 is closed. Processor 40 (FIG. 2) may control operation of switch 78 and control unit 56 via one or more enable signals sent via data bus 46 (FIG. 2). In other instances, control unit 56 may receive the enable signal from processor 40 and control operation of switch 78 and switch 54.

In the reflectance ultrasound mode, control unit 56 selectively opens and closes switch 54 to adjust the reflectance of ultrasound transducer 52 to modulate the reflected portion of the carrier signal with data for transmission to external device 18. Ultrasound transducer 52, switch 54 and control unit 56 operate in the same manner as described in detail with respect to FIG. 3. Therefore, a detailed description of their operation will not be described here. Telemetry module 70 may operate in the reflectance communication mode when unidirectional communication is sufficient, as is the case when a programming device or monitoring device simply downloads real-time or stored data from IMD 20.

However, in instances in which IMD 20 needs to receive data, e.g., as in the case of programming IMD 20, telemetry module 70 may operate in the direct pulsed communication mode. In the direct pulsed mode, telemetry module 70 receives modulated ultrasound carrier signals from another device, such as external device 18. In particular, ultrasound transducer 52 receives the modulated ultrasound carrier signal and converts the signal to a modulated electrical signal. The modulated electrical signal is provided to demodulator 72, which demodulates the signal to obtain the data transmitted by external device 18. Demodulator 72 provides the data to processor 40, which may process the data or store the data in memory 42 for later processing.

In the direct pulsed ultrasound mode, telemetry module 70 may also transmit outbound modulated ultrasound signals. In particular, signal generator 76 generates a carrier signal. The carrier signal may, for example, be a square wave carrier signal. However, other types of carrier signals may be used instead of a square wave. Modulator 74 modulates the carrier signal from signal generator 76 with outbound data to be transmitted. Modulator 74 may receive the data from processor 40 via data bus 46 (FIG. 2). Modulator 74 drives ultrasound transducer 52 with the modulated electrical carrier signal to produce a modulated ultrasound carrier signal for transmission to another device, such as external device 18. In some instances, modulator 74 and demodulator 72 may comprise a single modulator-demodulator (MODEM) component. Thus, in some instances, telemetry module 70 may transmit and receive data in the direct pulsed mode.

In other instances, telemetry module 70 may operate in the direct pulsed mode only for receiving data. For example, telemetry module 70 may utilize the reflectance mode for transmitting data and the direct pulsed mode for receiving data. In this case, switch 78 may be open during transmit cycles, i.e., periods of time designated for telemetry module 70 to transmit data, and control unit 56 selectively opens and closes switch 54 to adjust the reflectance of ultrasound transducer 52 to modulate the reflected portion of the carrier signal with data for transmission to external device 18. Switch 78 may be closed and switch 54 opened during the receive cycles, i.e., periods of time designated for telemetry module 70 to receive data, and the modulated signals received by transducer may be demodulated by demodulator 72 and provided to processor 40. In this case, telemetry module 70 may not include modulator 74 and signal generator 76.

In other instances, IMD 20 and external device 18 may communicate using a full duplex communication channel in which communications from external device 18 to IMD 20 (download communications) are modulated using a first modulation technique and the communications from IMD 20 to external device 18 (upload communications) are modulated using the reflectance mode with a second modulation technique that is different than the first modulation technique. For example, transducer 52 may receive a frequency modulated signal from external device 18 and demodulator 72 may demodulate the signal to obtain the data transmitted by external device 18. Transducer 52 still reflects a portion of the carrier signal incident on the transducer. Telemetry module 70 may adjust the reflectance of the transducer to amplitude modulate the reflected portion of the carrier signal. In this manner, telemetry module 70 may be capable of full duplex communication with external device 18. In other examples, upload communications may be modulated using phase modulation and download communication may be modulated using amplitude modulation, upload communications may be modulated using amplitude modulation and download communication may be modulated using frequency modulation (e.g., by selectively applying a non-resistive load), or other combinations of modulation techniques.

To further reduce power consumption, telemetry module 70 may power down components when not in use. For example, telemetry module 70 may power down modulator 74, demodulator 72 and/or signal generator 76 when operating in the ultrasound reflectance communication mode.

Telemetry module 70 may be useful in situations in which it is desirable to have bidirectional communication. For example, bidirectional communication may be desired for configuring operation of IMD 20, such as programming IMD 20 to provide therapy in accordance with a selected therapy program, programming IMD 20 to acquire physiological data, and/or programming IMD 20 to perform one or more diagnostic tests (e.g., battery life test or lead integrity test). In other instances, it is not practical to drive one of the transducers with a continuous electrical signal. In the case of intra-body communication between IMD 20 and another IMD, it is not practical to drive a transducer of one of the IMDs with a continuous electrical signal due to the limited supply of power.

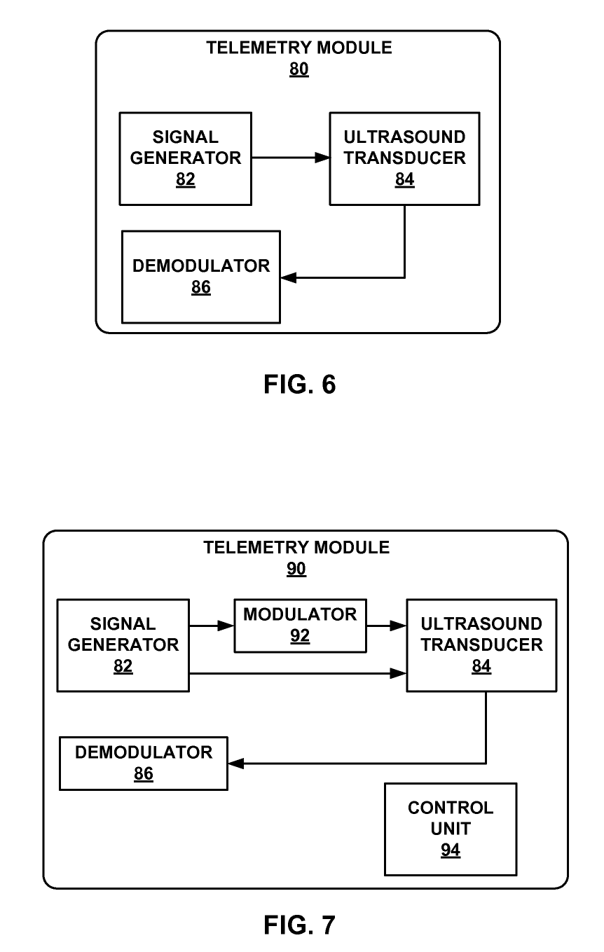

FIG. 6 is a block diagram illustrating an example telemetry module 80 that retrieves data from an IMD, such as IMD 20, in accordance with the ultrasound reflectance communication techniques of this disclosure. Telemetry module 80 may correspond to telemetry module 22 of external device 18 illustrated in FIG. 2. Telemetry module 80 includes a signal generator 82, an ultrasound transducer 84 and a demodulator 86.

Signal generator 82 generates a continuous electrical carrier signal and drives ultrasound transducer 84 with the continuous electrical carrier signal. The continuous electrical carrier signal is not modulated by telemetry module 80. Instead, the continuous electrical carrier signal is provided directly to ultrasound transducer 84, which converts the electrical carrier signal into an unmodulated ultrasound carrier signal for transmission to IMD 20. The continuous electrical carrier signal generated by signal generator 82 may be a continuous electrical signal, such as a continuous sine wave. The frequency of the continuous electrical signal generated by signal generator 82 may be between serial resonance and a parallel resonance of ultrasound transducer 84, which in one example may range from 1 to 10 megahertz (MHz).

As described in detail above, IMD 20 selectively adjusts the reflectance of its ultrasound transducer to modulate the reflected portion of the carrier signal with outbound data. Ultrasound transducer 74 detects the reflected portion of the carrier signal that has been modulated with the data from IMD 20 and converts the reflected portion of the carrier signal into a modulated electrical carrier signal. Demodulator 86 demodulates the modulated signal to obtain the data from IMD 20. Demodulator 86 may provide the demodulated signal to processor 26 (FIG. 2), which may process the data or store the retrieved data in memory 28 for later processing or later transmission to another device, such as a remote server. In the example illustrated in FIG. 6, ultrasound transducer 84 transmits the unmodulated carrier signal and receives the reflected portion of the carrier signal modulated with data. In other instances, however, telemetry module 80 may include more than one transducer, such as a first ultrasound transducer for transmitting data and a second ultrasound transducer for receiving data.

FIG. 7 is a block diagram illustrating another example telemetry module 90. Telemetry module 90 may correspond to telemetry module 22 of external device 18 illustrated in FIG. 2. Telemetry module 90 is capable of communicating in at least two different ultrasound communication modes. Telemetry module 90 may, in one example, operate in a reflectance ultrasound mode and a direct pulsed ultrasound mode. Telemetry module 90 includes a signal generator 82, an ultrasound transducer 84, a demodulator 86, a modulator 92 and a control unit 94.

In the reflectance ultrasound communication mode, telemetry module 90 operates in the same manner described above with respect to telemetry module 80. In particular, signal generator 82 provides a continuous electrical carrier signal to ultrasound transducer 84, which converts the carrier signal into an unmodulated ultrasound carrier signal for transmission to IMD 20. Additionally, ultrasound transducer 74 detects the reflected portion of the carrier signal that has been modulated with the data by IMD 20 and converts the reflected portion of the carrier signal into a modulated electrical carrier signal. Demodulator 86 demodulates the modulated signal and provides the demodulated signal to processor 26 (FIG. 2). When operating in the reflectance ultrasound communication mode, control unit 94 may power down at least a portion of the circuitry of modulator 92 since it is not being used. Telemetry module 90 may operate in the reflectance communication mode when unidirectional communication is sufficient, as is the case when a device including telemetry module 90 (e.g., external device 18) simply downloads real-time or stored data from IMD 20.

However, in instances in which the device including telemetry module 90 (e.g., external device 18) needs to transmit data to IMD 20, e.g., as in the case of programming IMD 20, telemetry module 90 may operate in the direct pulsed communication mode. In the direct pulsed ultrasound mode, telemetry module 90 may transmit outbound modulated ultrasound signals instead of a continuous unmodulated ultrasound signal. In particular, signal generator 82 generates a carrier signal. The carrier signal may, for example, be a square wave carrier signal. Other types of carrier signals may be used instead of a square wave. Modulator 92 modulates the carrier signal from signal generator 82 with outbound data to be transmitted. Modulator 92 may receive the data from processor 26 via data bus 32 (FIG. 2). Modulator 92 drives ultrasound transducer 84 with the modulated electrical carrier signal to produce a modulated ultrasound carrier signal for transmission to another device, such as IMD 20.

In the direct pulsed mode, telemetry module 90 may also receive modulated ultrasound carrier signals from another device, such as IMD 20. In this manner, the direct pulsed communication mode enables bidirectional communication with the other device. In particular, ultrasound transducer 52 receives the modulated ultrasound carrier signal and converts the signal to a modulated electrical signal. The modulated electrical signal is provided to demodulator 86, which demodulates the signal to obtain the data transmitted by IMD 20. Demodulator 86 provides the data to processor 26, which may process the data or store the data in memory 28 for later processing. In some instances, modulator 92 and demodulator 86 may comprise a single modulator-demodulator (MODEM) component.

In other instances, telemetry module 90 may operate in the reflectance mode for retrieving data from IMD 20 and operate in the direct pulsed mode for transmitting outbound data to IMD 20. In this case, control unit 94 controls telemetry module 90 to generate the modulated carrier signal during transmit cycles, i.e., periods of time designated for telemetry module 90 to transmit data, and controls telemetry module 90 to generate a continuous, unmodulated carrier signal during the receive cycles, i.e., periods of time designated for telemetry module 90 to retrieve data from IMD 20.

In a further embodiment, telemetry module 90 may communicate using a full duplex communication channel in which communications from external device 18 to IMD 20 (download communications) are modulated using a first modulation technique and the communications from IMD 20 to external device 18 (upload communications) are modulated using the reflectance mode with a second modulation technique that is different than the first modulation technique. For example, signal generator 82 generates a carrier signal and modulator 92 modulates the carrier signal using a first modulation technique, e.g., frequency modulation for purposes of illustration. Ultrasound transducer 84 transmits a frequency modulated ultrasound carrier signal to IMD 20.

Ultrasound transducer 84 detects the reflected portion of the carrier signal that has been modulated with the data by IMD 20 using a second modulation technique, e.g., amplitude modulation for purposes of example. Transducer 84 converts the reflected portion of the carrier signal into a modulated electrical carrier signal and demodulator 86 demodulates the modulated signal and provides the demodulated signal to processor 26 (FIG. 2). In this manner a full duplex acoustic communication system may be realized. In other examples, telemetry module 90 may transmit signals modulated using phase modulation and receive signals modulated using amplitude modulation, transmit signals modulated using amplitude modulation and receive signals modulated using frequency modulation, or other combinations of modulation techniques.

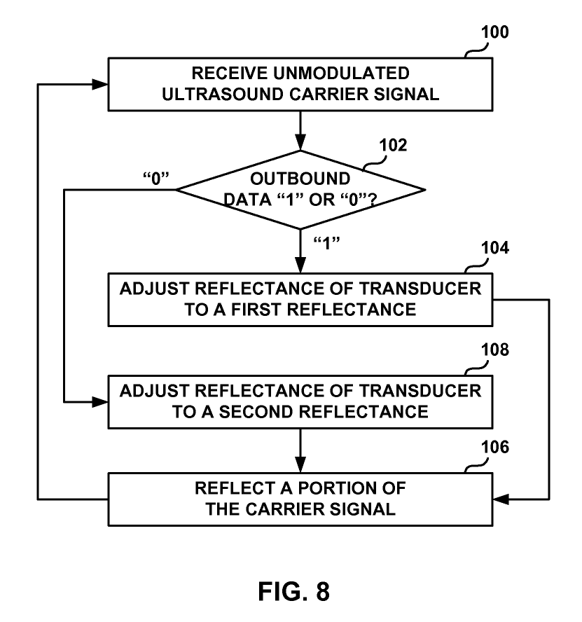

FIG. 8 is a flow diagram illustrating example operation of a telemetry module of an IMD operating in accordance with the ultrasound reflectance communication mode. The telemetry module may be any of telemetry modules 20, 50, 60 or 70. Initially, ultrasound transducer 52 receives an unmodulated carrier signal from another device, such as external device 18, incident on ultrasound transducer 52 (100).

Control unit 56 determines whether the outbound data is a “1” or a “0” (102). If the outbound data is a “1,” control unit 56 adjusts a reflectance of ultrasound transducer 52 to a first reflectance (104). Control unit 56 may, for example, adjust the reflectance of ultrasound transducer 52 by closing a switch 54 connected between electrical terminals of ultrasound transducer 52. Ultrasound transducer 52 reflects a portion of the incident ultrasound carrier signal (106). The amount of the ultrasound carrier signal that is reflected is dependent upon the reflectance.

If the outbound data is a “0,” control unit 56 adjusts a reflectance of ultrasound transducer 52 to a second reflectance (108). Control unit 56 may, for example, adjust the reflectance of ultrasound transducer 52 by opening a switch 54 connected between electrical terminals of ultrasound transducer 52. The second reflectance is either higher or lower than the first reflectance. Whether it is higher or lower does not matter as long as it is significantly different. Ultrasound transducer 52 reflects a portion of the incident ultrasound carrier signal (106). Because the amount of the ultrasound carrier signal that is reflected is dependent upon the reflectance the reflected portion of the carrier signal will be amplitude modulated with the information to be transmitted.

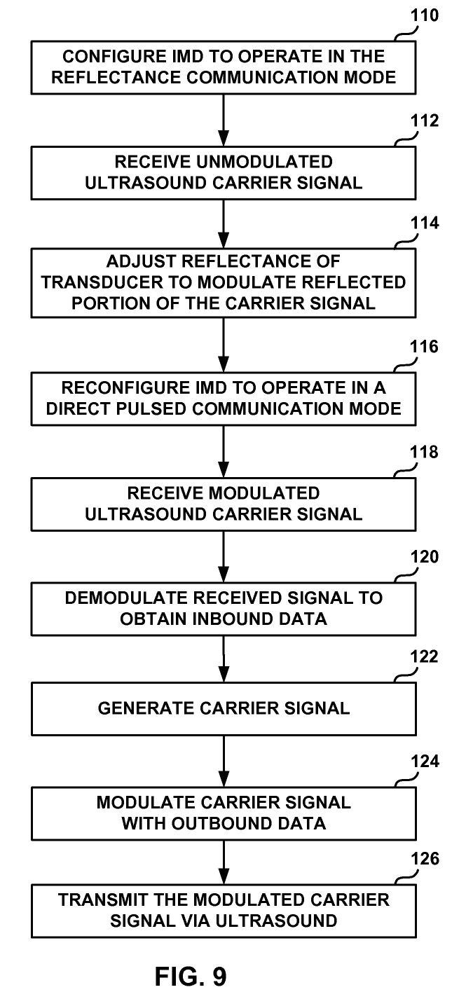

FIG. 9 is a flow diagram illustrating example operation of an IMD communicating using ultrasound communication in accordance with one aspect of this disclosure. Initially, IMD is configured to operate in a reflectance ultrasound communication mode (110). In the example illustrated in FIG. 5, for example, switch 78 may be opened to operate in the reflectance communication mode. In the reflectance ultrasound mode, ultrasound transducer 52 receives an unmodulated ultrasound carrier signal incident on ultrasound transducer 52 (112) and adjusts the reflectance of ultrasound transducer 52 to modulate the reflected portion of the carrier signal with data for transmission to external device 18 (114).

IMD may be reconfigured to operate in direct pulsed ultrasound communication mode (116). Control unit 56 may, for example, open switch 54 and close switch 78 to operate in the direct pulsed communication mode. In the direct pulsed mode, telemetry module 70 receives a modulated ultrasound carrier signal from another device, such as external device 18 (118). Demodulator 72 demodulates the signal to obtain the data transmitted by external device 18 (120). Demodulator 72 provides the data to processor 40, which may process the data or store the data in memory 42 for later processing.

In the direct pulsed ultrasound mode, telemetry module 70 may also transmit outbound modulated ultrasound signals. In particular, signal generator 76 generates a carrier signal (122). Modulator 74 modulates the carrier signal from signal generator 76 with outbound data to be transmitted (124). Ultrasound transducer 52 transmits the modulated carrier signal via ultrasound (126). For example, ultrasound transducer 52 converts the modulated electrical signal from modulator 74 to a modulated ultrasound carrier signal for transmission to another device. In some instances, telemetry module 70 may operate in the direct pulsed mode only for receiving data. For example, telemetry module 70 may utilize the reflectance mode for transmitting data and the direct pulsed mode for receiving data. In this case, the IMD may not perform step 122-126.

The techniques described in this disclosure may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, or other devices. The term “processor” or “processing circuitry” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. The described examples, however, should not be viewed as limiting of the techniques. For example, although primarily described in the context of communicating using ultrasound signals, the techniques of this disclosure may be used for communicating using other acoustic signals. Moreover, the techniques may be extended for communicating using non-acoustic signals for which a portion of the incident signal is reflected. These and other examples are within the scope of the following claims.

1. An implantable medical device comprising:
a transducer that receives an acoustic carrier signal incident on the transducer; and
a control unit that modulates a reflected portion of the acoustic carrier signal with data to be transmitted from the implantable medical device.

2. The device of claim 1, wherein the control unit selectively adjusts a reflectance of the transducer to modulate the reflected portion of the carrier signal.

3. The device of claim 2, further comprising a switch that is connected between electrical terminals of the transducer, wherein the control unit opens the switch to provide the transducer with a first reflectance and closes the switch to provide the transducer with a second reflectance.

4. The device of claim 3, wherein
the switch comprises a transistor, and
the control unit causes the transistor to operate in an OFF state to provide the first reflectance and causes the transistor to operate in an ON state to provide the second reflectance.

5. The device of claim 3, wherein the switch comprises an electromechanical switch.

6. The device of claim 3, further comprising a power source that collects the energy dissipated while the switch is closed.

7. The device of claim 1, wherein the control unit selectively applies a non-resistive load to electrical terminals of the transducer to frequency modulate the reflected portion of the carrier signal.

8. The device of claim 1, further comprising a demodulator, wherein
the control unit modulates the reflected portion of the acoustic carrier signal in a first communication mode, and
in a second communication mode, the demodulator demodulates an electrical signal output by the transducer to obtain data transmitted from another device.

9. The device of claim 8, wherein the device operates in the first communication mode during transmit cycles and operates in the second communication mode during receive cycles.

10. The device of claim 8, further comprising:
a signal generator that generates electrical carrier signals; and
a modulator that modulates the electrical carrier signals of the signal generator with data for transmission from the implantable medical device, wherein the transducer converts the electrical signal modulated with the data into an acoustic signal modulated with the data and transmits the acoustic signal in the second communication mode.

11. The device of claim 8, wherein the transducer receives an unmodulated acoustic carrier signal in the first communication mode and receives a modulated acoustic carrier signal in the second communication mode.

12. The device of claim 1, further comprising a demodulator, wherein
the transducer receives an acoustic carrier signal modulated with data using a first modulation technique,
the demodulator demodulates an electrical signal output by the transducer to obtain data transmitted from another device, and
the control unit modulates, using a second modulation technique that is different than the first modulation technique, a reflected portion of the acoustic carrier signal with data to be transmitted from the implantable medical device.

13. The device of claim 1, wherein the acoustic signal comprises an ultrasound signal.

14. A method comprising:
receiving an acoustic carrier signal incident on a transducer of an implantable medical device; and
modulating a reflected portion of the acoustic carrier signal with data to be transmitted from the implantable medical device.

15. The method of claim 14, wherein modulating a reflected portion of the acoustic carrier signal with data to be transmitted from the implantable medical device comprises selectively adjusting a reflectance of the transducer to modulate the reflected portion of the acoustic carrier signal with the data.

16. The method of claim 15, wherein selectively adjusting a reflectance of the transducer to modulate the reflected portion of the acoustic carrier signal with the data comprises opening a switch that is connected between electrical terminals of the transducer to provide the transducer with a first reflectance and closing the switch to provide the transducer with a second reflectance.

17. The method of claim 16, wherein
opening the switch comprises turning off a transistor to provide the transducer with the first reflectance; and
closing the switch comprises turning on the transistor to provide the transducer with the second reflectance.

18. The method of claim 16, further comprising collecting the energy dissipated when the switch is closed.

19. The method of claim 14, wherein modulating a reflected portion of the acoustic carrier signal with data to be transmitted from the implantable medical device comprises selectively applying a non-resistive load to electrical terminals of the transducer to frequency modulate the reflected portion of the carrier signal.

20. The method of claim 14, wherein modulating the reflected portion of the acoustic carrier signal comprises operating in a first communication mode by modulating the reflected portion of the acoustic carrier signal, the method further comprising:
operating in a second communication mode that includes:
receiving a modulated carrier signal incident on the transducer; and
demodulating an electrical signal output by the transducer to obtain data transmitted from another device.

21. The method of claim 20, wherein
operating in the first communication mode comprises operating in the first communication mode during transmit cycles, and
operating in the second communication mode comprises operating in the second communication mode during receive cycles.

22. The method of claim 20, further comprising, while operating in the second communication mode:
generating an electrical carrier signal;
modulating the generated electrical carrier signal with data for transmission from the device; and
converting the electrical signal modulated with the data into an acoustic signal modulated with the data; and
transmitting the acoustic signal modulated with the data.

23. The method of claim 14, wherein receiving the acoustic carrier signal comprises receiving an unmodulated acoustic carrier signal.

24. The method of claim 14, wherein:
receiving the acoustic carrier signal comprises receiving an acoustic carrier signal modulated with data using a first modulation technique, and
modulating the reflected portion of the carrier signal comprises modulating the reflected portion of the carrier signal using a second modulation technique that is different than the first modulation technique.

25. The method of claim 14, wherein the acoustic signal comprises an ultrasound signal.

26. An implantable medical device comprising:
means for receiving an acoustic carrier signal; and
means for modulating a reflected portion of the acoustic carrier signal with data to be transmitted from the implantable medical device.

27. The device of claim 26, wherein the modulating means selectively adjusts a reflectance of the receiving means to modulate the reflected portion of the acoustic carrier signal with the data.

28. The device of claim 27, wherein the modulating means opens a switch that is connected between electrical terminals of the receiving means to provide a first reflectance and closes the switch to provide a second reflectance.

29. The device of claim 26, wherein the modulating means selectively applyies a non-resistive load to electrical terminals of the receiving means to frequency modulate the reflected portion of the carrier signal.

30. The device of claim 26, further comprising means for controlling a communication mode of the device, wherein the controlling means
places the device in a first communication mode in which the receiving means receives an unmodulated carrier signal and the modulating means modulates a reflected portion of the acoustic carrier signal modulating the reflected portion of the acoustic carrier signal, and
places the device in a second operating mode in which the receiving means receives a modulated carrier signal and means for demodulating an electrical signal output by the receiving means demodulates the electrical signal to obtain data transmitted from another device.

31. The device of claim 30, wherein the controlling means
places the device in the first communication mode during transmit cycles, and
places the device in the second communication mode during receive cycles.

32. The device of claim 30, further comprising, while operating in the second communication mode:
means for generating an electrical carrier signal;
second means for modulating the generated electrical carrier signal with data for transmission from the device;
means for converting the electrical signal modulated with the data into an acoustic signal modulated with the data; and
means for transmitting the acoustic signal modulated with the data.

33. The device of claim 26, wherein:
the receiving means receives an acoustic carrier signal modulated with data using a first modulation technique, and
the modulating means modulates the reflected portion of the carrier signal using a second modulation technique that is different than the first modulation technique.
